(19) United States
(12) Patent Application Publication
(10) Pub. No.: US 2005/0208135 A1
(43) Pub. Date: Sep. 22, 2005

(54) Monocompartment osmotic controlled drug delivery system

(76) Inventors: Narayanan Badri Viswanathan, Tamil Nadu (IN); Ramakrishnan Sankar, Tamil Nadu (IN); Rajan Kumar Verma, New Delhi (IN); Rajeev Singh Raghuvanshi, New Delhi (IN); Ashok Kumar Rampal, Amristar (IN)

Correspondence Address: RANBAXY INC., 600 COLLEGE ROAD EAST, SUITE 2100, PRINCETON, NJ, 08540, US

(21) Appl. No.: 10/513,537

(22) PCT Filed: May 6, 2003
(86) PCT No.: PCT/IB03/01771

§ 371 Date: May 9, 2005

(30) Foreign Application Priority Data
May 6, 2002 (IN) 530/DEL/2002

(51) Int. Cl. A61K 9/24

(52) U.S. Cl. 424/473

(57) ABSTRACT

The present invention relates to a monocompartment osmotic controlled drug delivery system comprising a poorly soluble drug and at least one alginic acid derivative.

### FIELD OF THE INVENTION

The present invention relates to a monocompartment osmotic controlled drug delivery system comprising a poorly soluble drug and at least one alginic acid derivative.

### BACKGROUND OF THE INVENTION

Advantages of controlled release drug delivery systems are well documented. Numerous technologies have been exploited to achieve desired drug release profiles, as required to satisfy therapeutic needs and patient compliance. One such widely used controlled release technology is based on osmotic pressure controlled drug delivery, introduced by F. Theewas in J. Pharm. Sci., Vol. 64, 12, 1987-91 (1975). The elementary oral osmotic system (OROS®, Alza Corp.) in its simplest version takes the form of a conventional coated tablet. It comprises a homogenous core tablet of drug coated with a semi-permeable wall/layer and an aperture created through the wall for the release of contents from the core. When placed in dissolution media/gastrointestinal fluid, water permeates into the core through the semipermeable wall and dissolves the drug. The osmotic pressure thus built exerts pressure against the wall and thereby releases out the solution of drug through the aperture in the wall.

Osmotic controlled drug delivery systems show better in vitro-in vivo correlation as their performance is reported to be independent of pH and contents of the gastrointestinal tract. Moreover, they are highly resistant to mechanical stress encountered within the gut. Hence, properly designed osmotic systems may prove to be of paramount importance.

Use of simple osmotic system designed by F. Theewas is confined to a limited number of drugs, which are soluble enough to produce a sufficiently high osmotic pressure. Sparingly soluble drugs fail to be delivered from this system in the desired manner, and therefore demand skillful modifications of the Theewas design to exploit the advantages of these osmotic delivery systems.

U.S. Pat. No. 4,111,202 assigned to Alza Corp. addresses this problem by the fabrication of a “push-pull” (double compartment core) osmotic system wherein the core of the OROS® system, described above, is replaced by a pull compartment containing a sparingly soluble drug composition and a push compartment containing water soluble osmotically active agents. The two compartments are separated by means of an elastic diaphragm. When in operation, the osmotic pressure that builds up in the push compartment causes an increase in its volume. This increase in volume expands the elastic diaphragm, which thereby forces the drug out of the pull compartment through an aperture. Though advantageous over the OROS® system, manufacturing of “push-pull” systems is technically complicated and costly, requiring proper placement of elastic diaphragm between the two compartments. Further, for sparingly soluble drugs having large therapeutic doses, unacceptably large sized “push-pull” systems are needed.

The concept of “push-pull” systems is further simplified, as described in European Patent Application No. 52917, by developing osmotic systems without the elastic diaphragm. The osmotic system disclosed in this patent application has the two compartments of the push pull system replaced by two different composition layers, viz., drug layer containing drug and osmotic agents, and an expandable driving member layer formed of a water swellable hydrogel that absorbs fluid imbibed into the compartment and expands from a rested to an expanded state. The expansion of the driving member exerts pressure on the drug layer forcing its content out of the aperture. Manufacturing of the above system is still problematic, requiring multiple compression steps and a high level of uniformity in the grain size of granulate during compression. Identification of drug layer surface for drilling of aperture through the semipermeable wall is also cumbersome.

The above problems are overcome by a homogeneous monocompartment osmotic system disclosed in U.S. Pat. No. 4,857,336 reissued as Re 34,990 and U.S. Pat. No. 4,992,278, both assigned to Ciba-Geigy. U.S. Pat. No. 4,992,278 discloses a monocompartment therapeutic system comprising (a) a casing made of a material that is permeable to water and is impermeable to the components of the core containing the active ingredient; (b) a core containing an active ingredient that is sparingly soluble in water or a mixture of such active ingredients, a hydrophilic polymeric swelling agent consisting of a mixture of a vinylpyrrolidone/vinyl acetate copolymer with an ethylene oxide homopolymer, optionally water soluble substance for inducing osmosis and optionally further pharmaceutically acceptable adjuncts; and (c) passage through the casing (a) for the transport of the constituents contained in the core into the surrounding aqueous body fluid. Further, this patent teaches that the use of conventional swelling agents of two-compartment system such as polyvinylpyrrolidone, polyethylene oxide, polymethacrylate and the like, in single compartment system does not work. This is because the swelling pressure of these polymers is so great that in contact with water the semipermeable membrane bursts and the whole system disintegrates in the stomach after a short period of time.

There is thus a need for logical selection of a suitable swelling agent that enables easy fabrication of monocompartment system as well as provide controlled swelling without rupturing the semipermeable membrane. On the other hand, the swelling pressure should be sufficient enough to force the contents out of the system and achieve desired controlled drug release profiles.

### SUMMARY OF THE INVENTION

We have discovered that use of at least one alginic acid derivative as swelling agent in a monocompartment osmotic controlled drug delivery system overcomes the above problems and helps in achieving desired controlled drug release profiles for a poorly soluble drug.

In one general aspect, there is provided a monocompartment osmotic controlled drug delivery system that includes a poorly soluble drug and at least one alginic acid derivative.

Embodiments of the monocompartment osmotic controlled drug delivery system may include one or more of the following features. For example, the monocompartment osmotic controlled drug delivery system may further include a core, a semipermeable membrane enclosing at least a part of the core, and at least one passageway in the semipermeable membrane configured to deliver the contents of the core into the surrounding media. The core comprises the poorly soluble drug, the at least one alginic acid derivative, and at least one pharmaceutically acceptable inert excipient.

The core may further include an osmotic agent. The core may further include one or more additional layers below and/or above the semipermeable membrane. The one or more additional layers may include an immediate release layer of drug and the drug may be the same or different drug as in the core. The core may have a compact composition having a shape.

The poorly soluble drug may be a single drug or a combination of drugs. The poorly soluble drug may be one or more of an antidiabetic, antineoplastic agent, antihypertensive, psychopharmacological agent, cardiovascular agent, platelet aggregation inhibitor, analgesic, antimicrobial, diuretic, or spasmolytic. The poorly soluble drug may be one or more of glipizide, doxazosin, verapamil, prazosin, isradipine, cilostazol, nifedipine, nisoldipine, bendroflumethazide, chlorpropamide, hydrocortisone, ibuprofen, and diclofenac. The poorly soluble drug may be either of glipizide, doxazosin, or cilostazol. The poorly soluble drug may be glipizide present at either of approximately 2.5 mg, 5 mg or 10 mg.

The alginic acid derivative may be one or more of alginic acid and its pharmaceutically acceptable salts, pharmaceutically acceptable esters, or other pharmaceutically acceptable derivatives. The alginic acid salt may be one or more salts of alginic acid with sodium, potassium, magnesium, calcium or ammonia. The salt of alginic acid may be sodium alginate. The alginic acid ester may be propylene glycol alginate.

The pharmaceutically acceptable inert excipient may be one or more of binders, diluents, surfactants, pH modifiers, lubricants/glidants, stabilizers, plasticizers, and coloring agents.

The semipermeable membrane may be one or more of semipermeable membrane-forming polymers and coating additives. The semipermeable membrane-forming polymer may be one or more of cellulose derivatives, cellulose acetate, cellulose triacetate, agar acetate, amylose acetate, cellulose acetate ethyl carbamate, cellulose acetate phthalate, cellulose acetate methyl carbamate, cellulose acetate succinate, cellulose acetate dimethylaminoacetate, cellulose acetate ethyl carbonate, cellulose acetate chloroacetate, cellulose acetate ethyl oxalate, cellulose acetate methyl sulphonate, cellulose acetate butyl sulphonate, cellulose acetate propionate, cellulose acetate diethylamino-acetate, cellulose acetate octate, cellulose acetate laurate, cellulose acetate p-toluenesulphonate, cellulose acetate butyrate, polymeric epoxides, copolymers of alkylene oxides and alkyl glycidyl ethers, polyglycols, polylactic acid derivatives, and copolymers of acrylic acid ethyl ester and methacrylic acid methyl ester. The cellulose derivative may be cellulose acetate. The semipermeable membrane-forming polymer may be a combination of cellulose acetates having different degrees of acetylation.

The coating additives may be one or more of flux enhancers and pharmaceutically acceptable inert excipients. The flux enhancer may be one or more of hydroxymethyl cellulose, hydroxypropyl methylcellulose, polyethylene glycol, hydroxypropylcellulose, propylene glycol, and polyvinylpyrrolidone. The flux enhancer may be hydroxypropyl methylcellulose. The flux enhancer may be polyethylene glycol.

The osmotic agent may be one or more of water soluble salts of inorganic acids, water soluble salts of organic acids, non ionic organic compounds having high water solubility, water-soluble amino acids, urea, and urea derivatives. The one or more water soluble salts of inorganic acids may include magnesium chloride, magnesium sulfate, lithium chloride, sodium chloride, potassium chloride, lithium hydrogen phosphate, sodium hydrogen phosphate, potassium hydrogen phosphate, lithium dihydrogen phosphate, sodium dihydrogen phosphate, and potassium dihydrogen phosphate. The water soluble salts of organic acids may be one or more of sodium acetate, potassium acetate, magnesium succinate, sodium benzoate, sodium citrate, and sodium ascorbate. The non ionic organic compounds having high water solubility may be one or more carbohydrates, wherein carbohydrates includes one or more of mannitol, sorbitol, arabinose, ribose, xylose, glucose, fructose, mannose, galactose, sucrose, maltose, lactose, and raffinose. The water-soluble amino acids may be one or more of glycine, leucine, alanine, and methionine. The osmotic agent may be sorbitol or lactose.

In another general aspect, there is provided a process for preparing a monocompartment osmotic controlled drug delivery device includes
blending a poorly soluble drug, at least one alginic acid derivative, and at least one pharmaceutically acceptable inert excipient; and compressing the blend into a compact core;
enclosing the core with a solution/dispersion of an enclosing composition comprising one or more semipermeable membrane-forming polymers and other coating additives; and
forming at least one passageway in the semipermeable membrane.

Embodiments of the process may include one or more of the following features. For example, the process may further include granulating the blend with a binder before compressing the blend into a compact core. The process may further include blending at least one alginic acid derivative with the blend. The solution/dispersion of the enclosing composition may be made in a solvent that includes one or more of dichloromethane, isopropyl alcohol, acetone, methanol, ethanol, and water. The granulation may be made in a solvent that includes one or more of dichloromethane, isopropyl alcohol, acetone, methanol, ethanol, and water.

In another general aspect, a method of achieving controlled delivery of a poorly soluble drug over a period of at least 4 hours includes providing a monocompartment osmotic controlled drug delivery system comprising a poorly soluble drug and at least one alginic acid derivative.

The details of one or more embodiments of the invention are set forth in the accompanying drawings and the description below. Other features, objects, and advantages of the invention will be apparent from the description and the claims.

### BRIEF DESCRIPTION OF THE DRAWINGS

FIG. 1 is a graph that compares the in vitro release of drug (glipizide) from monocompartment osmotic controlled drug delivery systems as per the composition of Examples 1a, 1b and 1c.

FIG. 2 is a graph that compares the in vitro release of drug (glipizide) from monocompartment osmotic controlled drug delivery systems as per the composition of Examples 2a, 2b and 2c.

FIG. 3 is a graph that compares the in vitro release of drug (glipizide) from five different sets of monocompartment osmotic controlled drug delivery systems as per the compositions of Example 3, with semipermeable membrane thickness equivalent to weight gains of 11, 13, 15, 18 and 20% of core weight respectively.

FIG. 4 is a graph that compares the in vitro release of drug (doxazosin mesylate) from monocompartment osmotic controlled drug delivery systems as per composition of Examples 4a, 4b, 4c and 4d.

FIG. 5 is a graph that compares the in vitro release of drug (cilostazol) from monocompartment osmotic controlled drug delivery systems as per composition of Examples 5, with semipermeable membrane thickness equivalent to weight gains of 7.6 and 10.8% of core weight respectively.

### DESCRIPTION OF THE INVENTION

Alginic acid derivatives used as a swelling agent in the monocompartment osmotic controlled drug delivery system possess the required swelling property to form a dispersion of the poorly soluble drug of a consistency that is easily flowable through the passageway without damaging the semipermeable membrane. A combination of above attributes is rarely found amongst conventionally used swelling agents in osmotic systems. Moreover, the amount of alginic acid derivative used in the core may be varied over a wide range. Most of the alginic acid derivatives have been proven to be non-toxic to humans and other mammals on oral administration and are approved for human consumption. Further, with proper choice and use of a varying amount of osmotic agents and other pharmaceutically acceptable inert excipients, the drug delivery system may be designed to achieve drug release profiles of varied nature. The rate of drug release may also be manipulated by controlling the thickness and nature of semipermeable membrane, e.g., with a proper choice of other coating additives.

When the monocompartment osmotic controlled drug delivery system of the present invention is placed in dissolution media/gastrointestinal fluid, water permeates into the core, through the semipermeable membrane. Absorption of water causes swelling of the alginic acid derivative in the core, which thereby exerts pressure against the semipermeable membrane and forces the dispersion of poorly soluble drug through the passageway into the surrounding media. On coming out of the system, the drug in the dispersion is dissolved in the surrounding media.

The term “swelling” as used herein refers to an increase in the volume on coming in contact to water. In some cases swelling may even lead to a formation of a gel like consistency into which the poorly soluble drug is embedded in the form of dispersion. Hence, the terms “swelling” and “gelling” are used interchangeably herein.

The term “core” as used herein refer to and includes any compact composition having a defined shape such as tablet, mold, capsule and the like.

The term “poorly soluble drug” as used herein includes drugs having solubility of about 1 part in 25 or more parts of water. It also includes those drugs wherein 1 part of the drug dissolves is less than 25 parts of water, but under acidic or alkaline conditions, or under the influence of other excipients the solubility is decreased up to 1 in 25 parts of water. Suitable examples of the therapeutic classes, for the purpose of the present invention include antidiabetics, antineoplastic agents, antihypertensives, psychopharmacological agents, cardiovascular agents, platelet aggregation inhibitors, analgesics, antimicrobials, diuretics, spasmolytics and the like. Specific examples of poorly soluble drugs include glipizide, doxazosin, verapamil, prazosin, isradipine, cilostazol, nifedipine, nisoldipine, bendroflumethazide, chlorpropamide, hydrocortisone, ibuprofen, diclofenac, and the like, and combinations thereof. The term “drug” as used herein includes free drug well as any pharmaceutically acceptable salt thereof. The poorly soluble drug as used herein may be in a commercially available form as such; or in a processed form using techniques of comminution, micro emulsification, co-melting, solid dispersion, spray drying, co-processing with pharmaceutically acceptable inert excipients, drug-inclusion complexation and the like.

“Alginic acid derivative” as used herein include alginic acid as well as any of its pharmaceutically acceptable derivative such as salts, esters, and the like, and mixtures thereof. Specific examples of alginic acid salts include salts of alginic acid with sodium, potassium, magnesium, calcium or ammonia. Specific alginic acid esters include propylene glycol alginate.

Alginic acid is a naturally occurring hydrophilic colloidal polysaccharide consisting mainly of residues of β-1,4-linked D-mannuronic acid and α-1,4-liked L-glucuronic acid. Depending on the species of kelp used in manufacturing, ratios of mannuronic acid to glucuronic acid content typically range from 0.4 to 0.9. Alginic acid has an average molecular weight varying from about 10,000-6,00,000 and is widely used in the pharmaceutical field as a stabilizer, thickener, gelling agent and emulsifier. It is insoluble in water but its salts form thermally irreversible gels with water, whose viscosity decreases at higher pH values. Alginic acid derivatives are marketed by a white to yellowish brown filamentous, grainy, granular or powdered form under one or more of the nonexclusive list of the trade names—KELACID®, ALGINIC ACID HF/D, ALGINIC ACID DC, KELTONE® LVCR, KELTONE® HVCR, MANUCOL® LKX, MANUCOL LB, MANUCOL DMF, KELCOSOL®, MANUGEL® DMB, KELCOLOID® LVF, MANUCOL ESTER ERK, Improved KELMAR®, KELTOSE®. Based on the grade used and desired drug release profile, the amount of alginic acid derivative may vary from about 5% to about 98% by weight of the total weight of core.

One of the important factors in achieving effective hydration, and thereby controlled swelling of the alginic acid derivative, is proper dispersion of individual particles into the core. Poor dispersion may lead to the formation of large lumps of unhydrated alginic acid derivative and significantly extend the hydration and swelling time which can produce erratic drug release profiles.

One way of achieving proper dispersion of alginic acid derivative particles is blending with an osmotic agent, which diminishes its tendency to form lumps. Further, the osmotic agent may be used to manipulate the viscosity of the dispersion of poorly soluble drug formed in the core, and also to manipulate drug release profile.

The term “osmotic agent” as used herein includes all pharmaceutically acceptable inert water soluble compounds suitable for inducing osmosis as referred to in, for example, the Pharmacoepias, “Hager,” and Remington's Pharmaceutical Sciences. Examples of compounds suitable as osmotic agents include water soluble salts of inorganic acids such as magnesium chloride or magnesium sulfate, lithium chloride, sodium chloride, potassium chloride, lithium hydrogen phosphate, sodium hydrogen phosphate, potassium hydrogen phosphate, lithium dihydrogen phosphate, sodium dihydrogen phosphate, and potassium dihydrogen phosphate; water soluble salts of organic acids such as sodium acetate, potassium acetate, magnesium succinate, sodium benzoate, sodium citrate, and sodium ascorbate; non ionic organic compounds with high water solubility, e.g., carbohydrates such as mannitol, sorbitol, arabinose, ribose, xylose, glucose, fructose, mannose, galactose, sucrose, maltose, lactose, and raffinose; water-soluble amino acids such as glycine, leucine, alanine, and methionine; urea and urea derivatives; and the like and mixtures thereof. The amount of osmotic agent used in the core may be up to about 60% by weight of the total weight of core.

“Semipermeable membrane” as used herein is a membrane or coating that allows movement of water molecules through it but does not allow the contents of the core to pass through. The semipermeable membrane of the drug delivery system includes one or more membrane-forming polymers and other pharmaceutically acceptable coating additives. Membrane-forming polymers are those that are not metabolized in the gastrointestinal tract, i.e., are ejected unchanged from the body in feces. Membrane-forming polymers also include those known in the art for fabrication of semipermeable membrane and described in the literature, e.g., in U.S. Pat. Nos. 3,916,899 and 3,977,404. Examples of semipermeable membrane forming polymers include cellulose derivatives such as cellulose acetate, cellulose triacetate, agar acetate, amylose acetate, cellulose acetate ethyl carbamate, cellulose acetate phthalate, cellulose acetate methyl carbamate, cellulose acetate succinate, cellulose acetate dimethylaminoacetate, cellulose acetate ethyl carbonate, cellulose acetate chloroacetate, cellulose acetate ethyl oxalate, cellulose acetate methyl sulphonate, cellulose acetate butyl sulphonate, cellulose acetate propionate, cellulose acetate diethylamino-acetate, cellulose acetate octate, cellulose acetate laurate, cellulose acetate p-toluenesulphonate, and cellulose acetate butyrate; polymeric epoxides; copolymers of alkylene oxides and alkyl glycidyl ethers; polyglycols or polylactic acid derivatives; copolymer of acrylic acid ethyl ester and methacrylic acid methyl ester; and the like; and mixtures thereof. Alternatively, a combination of cellulose acetates with different degrees of acetylation may be used as membrane-forming polymer. As the degree of acetylation of cellulose acetate increases, permeability of the membrane decreases. In particular, a combination of cellulose acetates having acetyl content in the range of about 8% to about 50% may be used. Further, other coating additives may be combined with the membrane forming polymers to adjust the permeability as desired. Controlling membrane thickness also helps to manipulate the permeability of the membrane, which may vary from about 3% to about 40% weight build up over the weight of core.

The term “passageway” as used herein refers to and includes any suitable means for releasing the contents of the core into the surrounding media. The term includes passages, apertures, bores, holes, openings and the like, created through the semipermeable membrane and forming a connection between the core and the surrounding media. The passageway may be created by mechanical drilling or laser drilling, or formed in response to the osmotic pressure acting on the drug delivery system. Based on the nature of desired drug release profile, the number and diameter of the passageway may be adjusted. However, the diameter of the passageway should not be large enough to allow body fluids to enter the drug delivery system by the process of convection.

The term “pharmaceutically acceptable inert excipients” as used herein includes all excipients used in the art of manufacturing osmotic controlled dosage forms and described in the literature. Examples include binders, diluents, surfactants, pH modifiers, lubricants/glidants, stabilizers, plasticizers, coloring agents, and the like, and mixtures thereof.

Specific examples of binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, and the like, and mixtures thereof.

Specific examples of diluents include calcium carbonate, calcium phosphate-dibasic, calcium phosphate-tribasic, calcium sulfate, microcrystalline cellulose, powdered cellulose, dextrates, dextrins, dextrose excipients, fructose, kaolin, lactitol, lactose, marnitol, sorbitol, starch, pregelatinized starch, sucrose, compressible sugar, confectioners sugar, and the like, and mixtures thereof.

Surfactants may be used to promote wetting of poorly soluble drug as well as promote hydration of alginic acid derivative and include both non-ionic and ionic (cationic, anionic and witterionic) surfactants suitable for use in pharmaceutical compositions. These include polyethoxylated fatty acids and their derivatives, for example polyethylene glycol 400 distearate, polyethylene glycol-20 dioleate, polyethylene glycol 4-150 mono dilaurate, and polyethylene glycol-20 glyceryl stearate; alcohol-oil transesterification products, for example, polyethylene glycol—6 corn oil; polyglycerized fatty acids, for example, polyglyceryl—6 pentaoleate; propylene glycol fatty acid esters, for example, propylene glycol monocaprylate; mono and diglycerides, for example, glyceryl ricinoleate; sterol and sterol derivatives; sorbitan fatty acid esters and their derivatives, for example polyethylene glycol—20 sorbitan monooleate, and sorbitan monolaurate; polyethylene glycol alkyl ether or phenols, for example polyethylene glycol—20 cetyl ether, polyethylene glycol—10-100 nonyl phenol; sugar esters, for example, sucrose monopalmitate; polyoxyethylene-polyoxypropylene block copolymers known as “poloxamer”; ionic surfactants, for example, sodium caproate, sodium glycocholate, soy lecithin, sodium stearyl fumarate, propylene glycol alginate, octyl sulfosuccinate disodium, and palmitoyl carnitine; and the like; and mixtures thereof.

The pH modifiers are substances which help in maintaining the pH of the local environment surrounding the drug at a value favorable for suitably modifying the solubility behavior of drug and/or gelling behavior of alginic acid derivative. Specific examples of pH modifiers include dibasic sodium phosphate, sodium ascorbate, meglumine, sodium citrate, trimethanolamine, sodium hydroxide, potassium hydroxide, calcium hydroxide, magnesium oxide, magnesium hydroxide, ammonia, tertiary sodium phosphate, diethanolamine, ethylenediamine, L-lysine and the like, and mixtures thereof.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like, and mixtures thereof.

Specific examples of plasticizers include acetylated triacetin, triethylcitrate, tributylcitrate, glyceroltributyrate, monoglyceride, rape oil, olive oil, sesame oil, acetyltributylcitrate, acetyltriethylcitrate, glycerin sorbitol, diethyloxalate, diethyl phthalate, diethylmalate, diethylfumarate, dibutylsuccinate, diethylmalonate, dioctylphthalate, dibutylsebacate, and the like, and mixtures thereof.

Stabilizers include antioxidants, buffers, acids, and the like, and mixtures thereof.

Coloring agents include any FDA approved colors for oral use, and mixtures thereof.

The term “coating additives” as used herein includes all conventional coating additives used in the art of coating technology and described in the literature. Examples include flux enhancers as well as those described above under pharmaceutically acceptable inert excipients.

Flux enhancers are water soluble substances that aid in drawing water from the surrounding media and are thereby helpful in manipulating the semipermeable membrane's permeability. Specific examples include hydroxymethyl cellulose, hydroxypropyl methylcellulose, polyethylene glycol, hydroxypropylcellulose, propylene glycol, polyvinylpyrrolidone, and the like, and mixtures thereof.

In one of the embodiments, a monocompartment osmotic controlled drug delivery system is prepared by processes known in the prior art, e.g. by comminuting, mixing, granulation, sizing, filling, molding, spraying, immersing, coating etc. The core is prepared by: (1) blending a poorly soluble drug, at least one alginic acid derivative, optionally an osmotic agent and other pharmaceutically inert excipients; (2) optionally granulating the blend; and (3) compressing the blend/granules into a compact core. The compact core may be enclosed within a semipermeable membrane by applying the composition that forms the semipermeable in the form of a solution/dispersion. The solution or dispersion includes the polymer that forms the semipermeable membrane as well as coating additives. Finally, a passageway may be created through the semipermeable membrane using a suitable technique.

Examples of solvents used for the purpose of granulation or for preparing the solution/dispersion of the coating composition include dichloromethane, isopropyl alcohol, acetone, methanol, ethanol, water, and the like, and mixtures thereof.

Alternatively, additional coating layers may be applied over the cores either below and/or over the semipermeable membrane. The additional layers comprise coating additives and provide smooth surfaces over which the semipermeable membrane may be uniformly applied or identification marks may be printed. In addition, the layer or layers provide an aesthetic appeal.

If an immediate action is desired, the monocompartment osmotic controlled delivery system may be coated with an immediate release layer that includes the same drug as in the core, or a different drug over the semipermeable membrane.

Further, a combination of more than one drug may also be used in the core and/or in the immediate release layer. For example, if two or more drugs are prescribed to treat a condition and they are suitable for delivery in the monocompartment osmotic controlled delivery system, the drugs may be delivered in the core and/or the core and the immediate release layer.

The invention is further illustrated by the following examples, which are provided for illustrative purposes and should not be construed as limiting the scope of the invention.

### EXAMPLES 1a-1c

I. Core Composition

| | Weight (mg) per core | | |
|---|---|---|---|
| Ingredient | Example 1a | Example 1b | Example 1c |
| Glipizide | 11.0 | 11.0 | 10.0 |
| Sodium alginate | — | 137.5 | 100.0 |
| Sorbitol | 137.5 | — | 100.0 |
| Polyvinylpyrrolidone | 8.5 | 8.5 | 6.3 |
| Magnesium stearate | 2.0 | 2.0 | 2.2 |

II. Semipermeable Membrane Composition

| Ingredient | % by weight |
|---|---|
| Cellulose acetate (32% acetyl content) | 72.83 |
| Cellulose acetate (39.8% acetyl content) | 10.94 |
| Hydroxypropyl methylcellulose | 8.11 |
| Polyethylene glycol | 8.11 |

Procedure:
1. The core ingredients were sieved to the desired size level and the required amounts weighed out.
2. Glipizide, sodium alginate, sorbitol and polyvinylpyrrolidone were mixed together to form a homogenous blend.
3. The blend of step 2 was granulated using a mixture of isopropyl alcohol and methanol (50:50 v/v).
4. The wet granules were dried in a fluidized bed drier and sized through suitable sieves.
5. The dried granules were lubricated by blending with magnesium stearate and compressed into round concave shaped cores using suitable tooling.
6. Cellulose acetate, hydroxypropyl methylcellulose and polyethylene glycol were dissolved in a mixture of dichloromethane and methanol (80:20 w/w) to prepare a 4% w/w solution.
7. The cores of step 5 were coated with the solution of step 6 in a coating pan until they attained a weight gain of 10% (Example 1a and 1b) or 16% (Example 1c) of core weight.
8. The coated cores were dried in a hot air oven and then an orifice was drilled through the semipermeable membrane using a 1 mm mechanical drill to obtain monocompartment osmotic controlled drug delivery systems.

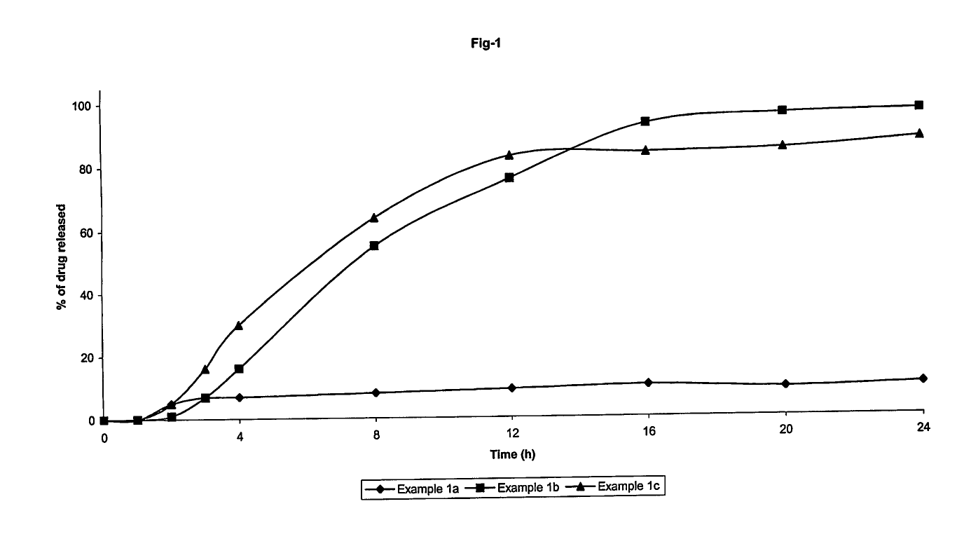

The in vitro release of drug (glipizide) from monocompartment osmotic controlled drug delivery systems as per Examples 1a, 1b and 1c was studied in 900 ml phosphate buffer (pH 7.5) using USP II dissolution apparatus, at a paddle speed of 50 rpm. The results of the study are illustrated in FIG. 1.

FIG. 1 demonstrates that both the rate and the amount of drug released for the composition of Example 1a (having no alginic acid derivative) were drastically lower than the rate and amount release for the compositions of Examples 1b and 1c. Hence, alginic acid derivatives play a major role in achieving acceptable release profiles for poorly soluble drugs from monocompartment osmotic controlled drug delivery systems. Further, although the drug release profiles of Example 1b and 1c are very similar, Example 1c having an osmotic agent) has a lower lag time than Example 1b. Thus, controlling the amount and use of an osmotic agent in combination with an alginic acid derivative is a useful approach in manipulating drug release profiles.

### EXAMPLES 2a-2c

I. Core Composition

| | Weight (mg) per core | | |
|---|---|---|---|
| Ingredient | Example 2a | Example 2b | Example 2c |
| Glipizide | 10.0 | 10.0 | 10.0 |
| Sodium alginate | 125.0 | 125.0 | 125.0 |
| Sorbitol | 125.0 | 62.5 | — |
| Lactose | — | 62.5 | 125.0 |
| Polyvinylpyrrolidone | 12.0 | 12.0 | 12.0 |
| Magnesium stearate | 3.0 | 3.0 | 3.0 |

II. Semipermeable Membrane Composition

| Ingredient | % by weight |
|---|---|
| Cellulose acetate (32% acetyl content) | 72.83 |
| Cellulose acetate (39.8% acetyl content) | 10.94 |
| Hydroxypropyl methylcellulose | 8.11 |
| Polyethylene glycol | 8.11 |

Procedure:
1. The core ingredients were sieved to the desired size level and the required amounts weighed out.
2. Glipizide, sodium alginate, sorbitol, lactose and polyvinylpyrrolidone were mixed together to form a homogenous blend.
3. The blend of step 2 was granulated using isopropyl alcohol.
4. The wet granules were dried in a fluidized bed drier and sized through suitable sieves.
5. The dried granules were lubricated by blending with magnesium stearate and compressed into round concave shaped cores using suitable tooling.
6. Cellulose acetate, hydroxypropyl methylcellulose and polyethylene glycol were dissolved in a mixture of dichloromethane and methanol (80:20 w/w) to prepare a 3.5% w/w solution.
7. The cores of step 5 were coated with the solution of step 6 in a coating pan until they attained a weight gain of 17% of core weight.
8. The coated cores were dried in a hot air oven and then an orifice was drilled through the semipermeable membrane using a 1 mm mechanical drill to obtain monocompartment osmotic controlled drug delivery systems.

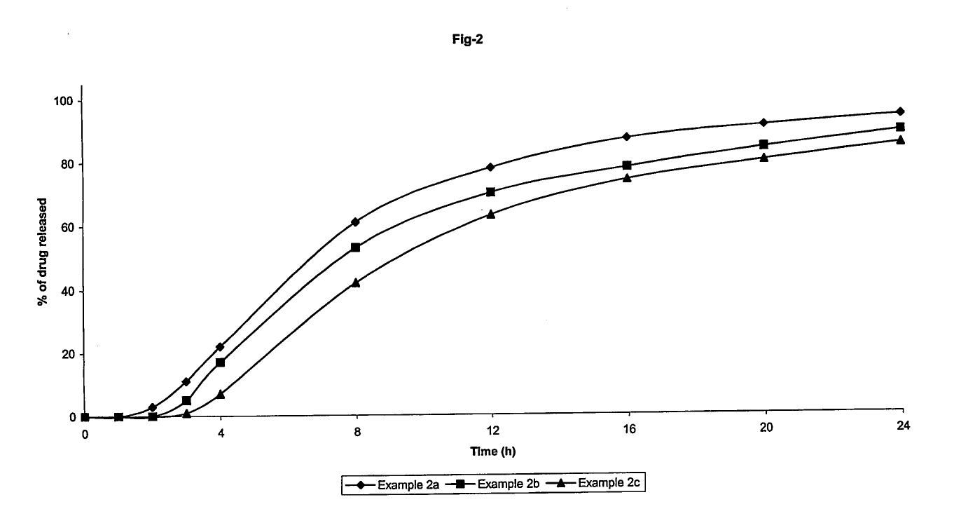

The in vitro release of drug (glipizide) from monocompartment osmotic controlled drug delivery systems as per Examples 2a, 2b, and 2c was studied in 900 ml phosphate buffer (pH 7.5) using USP II dissolution apparatus, at a paddle speed of 50 rpm. The results of the study are illustrated in FIG. 2.

FIG. 2 reveals that though the drug release profiles from Examples 2a, 2b, and 2c are very similar, the lag time for Example 2a (using sorbitol as osmotic agent) is lower than that obtained for Example 2b (using sorbitol and lactose in equal weights as osmotic agents), which is again lower than that obtained for Example 2c (using lactose as osmotic agent). As the solubility of lactose is much less than sorbitol, it can be inferred that with the increase in solubility of osmotic agent, lag time decreases. Hence, delivery systems with the desired lag time may be achieved by proper selection of osmotic agents.

### EXAMPLE 3

I. Core Composition

| Ingredient | Weight (mg) per core |
|---|---|
| Glipizide | 11.0 |
| Sodium alginate | 105.0 |
| Sorbitol | 170.0 |
| Colloidal silicon dioxide | 2.0 |
| Polyvinylpyrrolidone | 15.0 |
| Magnesium stearate | 6.0 |

II. Precoating Composition

| Ingredient | % by weight |
|---|---|
| Hydroxypropyl methylcellulose | 80.0 |
| Polyethylene glycol | 20.0 |

III. Semipermeable Membrane

| Ingredient | % by weight |
|---|---|
| Cellulose acetate (32% acetyl content) | 72.83 |
| Cellulose acetate (39.8% acetyl content) | 10.94 |
| Hydroxypropyl methylcellulose | 8.11 |
| Polyethylene glycol | 8.11 |

Procedure:
1. The core ingredients were sieved to the desired size level and the required amounts weighed out.
2. Glipizide, sodium alginate and sorbitol were mixed together to form a homogenous blend.
3. The blend of step 2 was granulated using a solution of polyvinylpyrrolidone in isopropyl alcohol.
4. The wet granules were dried in a fluidized bed drier and sized through suitable sieves.
5. The dried granules were lubricated by blending with magnesium stearate and compressed into round concave shaped cores using suitable tooling.
6. Hydroxypropyl methylcellulose and polyethylene glycol were dissolved in a mixture of isopropyl alcohol and dichloromethane (60:40 w/w) to prepare a 5% w/w solution.
7. The cores of step 5 were coated with the solution of step 6 in a coating pan to form a precoated core until they attained a weight gain of 1% of core weight.
8. Cellulose acetate, hydroxypropyl methylcellulose and polyethylene glycol were dissolved in a mixture of dichloromethane and methanol (80:20 w/w) to prepare a 3.5% w/w solution.
9. The precoated cores of step 7 were coated with the solution of step 8 in a coating pan to prepare five different sets of coated cores having weight gain of 11, 13, 15, 18 and 20% of core weight, respectively.
10. The coated cores of step 10 were dried in a hot air oven and then an orifice was drilled through the semipermeable membrane using a 0.6 mm mechanical drill to obtain monocompartment osmotic controlled drug delivery systems.

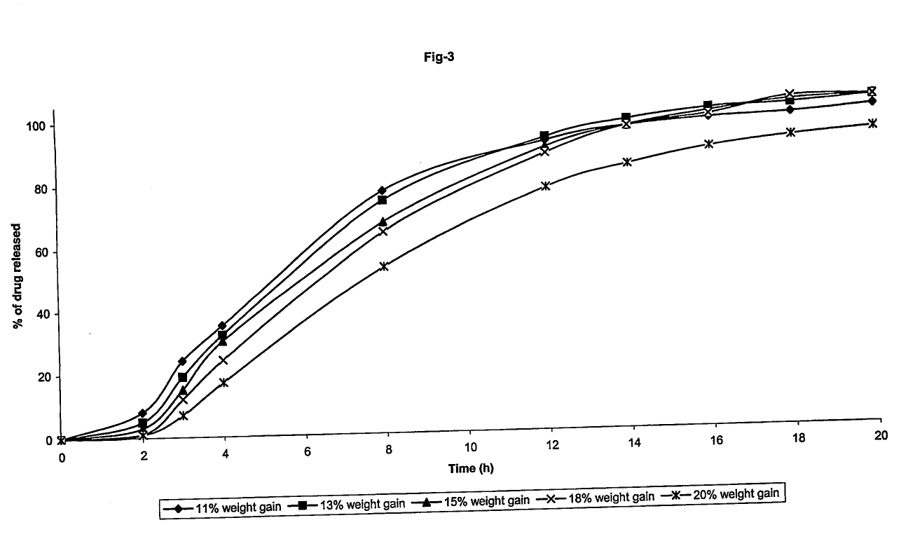

The in vitro release of drug (glipizide) from five different sets of monocompartment osmotic controlled drug delivery systems as per composition of Example 3 and with semipermeable membrane thickness equivalent to weight gain of 11, 13, 15, 18 and 20% of core weight respectively, was studied in 900 ml phosphate buffer (pH 7.5) using USP II dissolution apparatus, at a paddle speed of 50 rpm. The results of the study are illustrated in FIG. 3.

The drug release profiles in FIG. 3 clearly indicate a decrease in drug release rate with an increase in the semipermeable membrane thickness equivalent to above 18% weight gain of core weight. Hence, controlling the thickness of the semipermeable membrane can be used to manipulate the drug release profiles.

### EXAMPLE 4a-4d

I. Core Composition

Weight (mg) per core
Example No.

| Ingredient | 4a | 4b | 4c | 4d |
|---|---|---|---|---|
| Doxazosin mesylate | 10.7 | 10.7 | 10.7 | 10.7 |
| Sodium alginate | 135.0 | 100.0 | 100.0 | 137.3 |
| Sorbitol | 135.0 | 170.0 | 170.0 | 134.7 |
| Colloidal silicon | 2.0 | 2.0 | 2.0 | 2.6 |
| dioxide | | | | |
| Polyvinylpyrrolidone | 14.3 | 14.3 | 14.3 | 15.3 |
| Magnesium oxide | — | 30.0 | — | — |
| Meglumine | — | — | 30.0 | — |
| Poloxamer | — | — | — | 10.0 |
| Magnesium stearate | 3.0 | 3.0 | 3.0 | 4.6 |

II. Semipermeable Membrane Composition

% by weight

| Ingredient | Example 4a-4c | Example 4d |
|---|---|---|
| Cellulose acetate (39.8% | 77.0 | 83.9 |
| acetyl content) | | |
| Hydroxypropyl | — | 8.0 |
| methylcellulose | | |
| Polyethylene glycol 4000 | 11.5 | 8.0 |
| Polyethylene glycol 400 | 11.5 | — |

Procedure:
1. The core ingredients were sieved to the desired size level and the required amounts weighed out.
2. Doxazosin mesylate, sodium alginate, sorbitol, polyvinylpyrrolidone, colloidal silicon dioxide, magnesium oxide (Example 4b), meglumine (Example 4c) and polaxamer (Example 4d) were mixed together to form homogenous blends.
3. The blends of step 2 were lubricated by blending with magnesium stearate and compressed into round concave shaped cores using suitable tooling.
4. Cellulose acetate and polyethylene glycol were dissolved in a mixture of acetone and water (90:10 w/w) to prepare a 4% w/w solution (Example 4a-4c), whereas for Example 4d cellulose acetate, polyethylene glycol and hydroxypropyl methylcellulose were dissolved in a mixture of dichloromethane and methanol (80:20 w/w) to prepare a 3.5% w/w solution.
5. The cores of step 5 were coated with the corresponding coating solutions of step 6 in a coating pan until they attained a weight gain of 11% (Example 4a) or 12% (Example 4b, 4c and 4d) of core weight.
6. The coated cores were dried in a hot air oven and then an orifice was drilled through the semipermeable membrane using a 0.6 mm mechanical drill to obtain monocompartment osmotic controlled drug delivery systems.

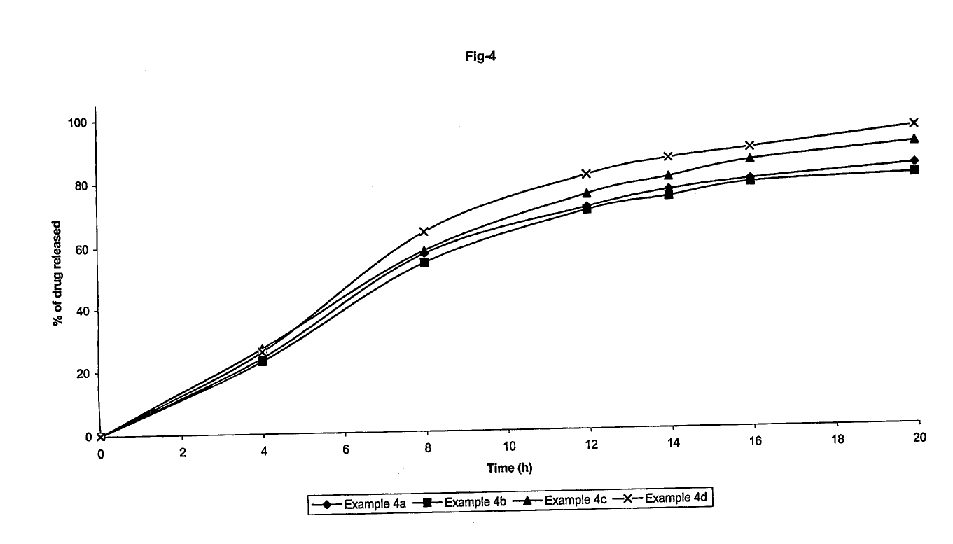

The in vitro release of drug (doxazosin mesylate) from monocompartment osmotic controlled drug delivery systems as per Examples 4a-4d was studied in 900 ml phosphate buffer (pH 6.8) with 0.5% sodium lauryl sulphate using USP II dissolution apparatus, at a paddle speed of 50 rpm. The results of the study are illustrated in FIG. 4.

### EXAMPLE 5

I. Core Composition

| Ingredient | Weight (mg) per core |
|---|---|
| Cilostazol | 200.0 |
| Sodium alginate | 80.0 |
| Sorbitol | 52.0 |
| Lactose | 50.0 |
| Polyvinylpyrrolidone | 15.0 |
| Sodium lauryl sulphate | 20.0 |
| Magnesium stearate | 3.0 |

II. Semipermeable Membrane

| Ingredient | % by weight |
|---|---|
| Cellulose acetate (39.8% acetyl content) | 74.07 |
| Polyethylene glycol 4000 | 14.82 |
| Polyethylene glycol 400 | 11.11 |

Procedure:
1. The core ingredients were sieved to the desired size level and the required amounts weighed out.
2. Cilostazol, sodium alginate, sorbitol, lactose, sodium lauryl sulphate and polyvinylpyrrolidone were mixed together to form a homogenous blend.
3. The blend of step 2 was granulated using isopropyl alcohol.
4. The wet granules of step 3 were dried and sieved through suitable sieves.
5. The dried granules were lubricated by blending with magnesium stearate and compressed into round concave shaped cores using suitable tooling.
6. Cellulose acetate and polyethylene glycol were dissolved in a mixture of acetone and water (90:10 w/w) to prepare a 4% w/w solution.
7. The cores of step 5 were coated with the solution of step 6 in a coating pan to prepare two different sets of coated cores having weight gains of 7.6% and 10.8% of core weight, respectively.
8. The coated cores of step 7 were dried in a hot air oven and then an orifice was drilled through the semipermeable membrane using a 0.6 mm mechanical drill to obtain monocompartment osmotic controlled drug delivery systems.

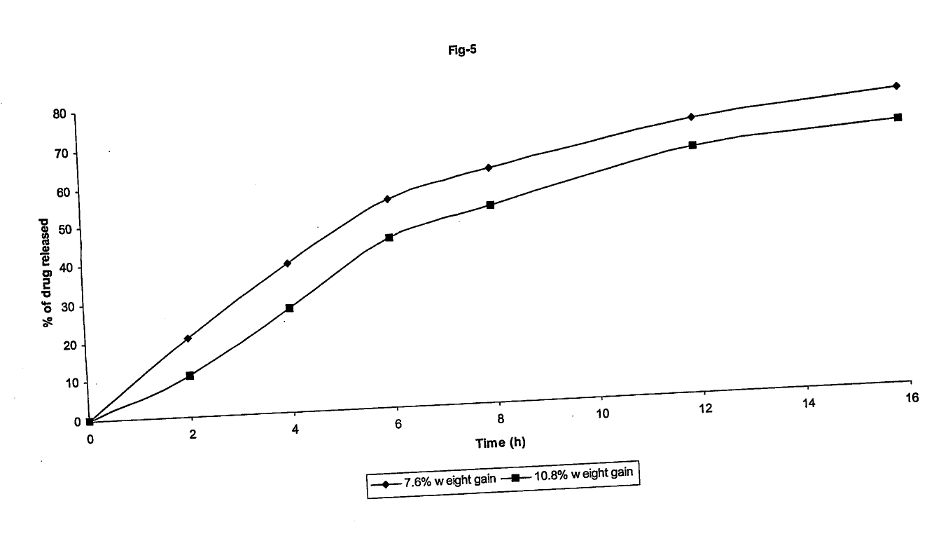

The in vitro release of drug (Cilostazol) from the two different sets of monocompartment osmotic controlled drug delivery systems as per the compositions of Example 5 and with semipermeable membrane thickness equivalent to weight gains of 7.6% and 10.8% of core weight, respectively, was studied in 900 ml phosphate buffer (pH 6.8) with 0.25% sodium lauryl sulphate using USP II dissolution apparatus, at a paddle speed of 50 rpm. The results of the study are illustrated in FIG. 5.

While several particular forms of the invention have been illustrated and described, it will be apparent that various modifications and combinations of the invention detailed in the text and claims can be made without departing from the spirit and scope of the invention. For example, each monocompartment osmotic controlled delivery system can include approximately 2.5 mg, 5 mg, or 10 mg of glipizide. Moreover, it is contemplated that any single feature or any combination of optional features of the inventive variations described herein may be specifically excluded from the claimed invention and be so described as a negative invention. Accordingly, it is not intended that the invention be limited, except as by the appended claims.

1. A monocompartment osmotic controlled drug delivery system comprising a poorly soluble drug and at least one alginic acid derivative.

2. The monocompartment osmotic controlled drug delivery system of claim 1, further comprising a core, a semipermeable membrane enclosing at least a part of the core, and at least one passageway in the semipermeable membrane configured to deliver the contents of the core into the surrounding media, wherein the core comprises the poorly soluble drug, the at least one alginic acid derivative, and at least one pharmaceutically acceptable inert excipient.

3. The monocompartment osmotic controlled drug delivery system of claim 2, wherein the core further comprises an osmotic agent.

4. The monocompartment osmotic controlled drug delivery system of claim 2, wherein the core comprises one or more additional layers below and/or above the semipermeable membrane.

5. The monocompartment osmotic controlled drug delivery system of claim 4, wherein the one or more additional layers comprise an immediate release layer of drug, wherein the drug comprises the same or different drug as in the core.

6. The monocompartment osmotic controlled drug delivery system of claim 2, wherein the core comprises a compact composition having a shape.

7. The monocompartment osmotic controlled drug delivery system of claim 1, wherein the drug comprises a single drug or a combination of drugs.

8. The monocompartment osmotic controlled drug delivery system of claim 2, wherein the poorly soluble drug comprises one or more of an antidiabetic, antineoplastic agent, antihypertensive, psychopharmacological agent, cardiovascular agent, platelet aggregation inhibitor, analgesic, antimicrobial, diuretic, or spasmolytic.

9. The monocompartment osmotic controlled drug delivery system of claim 8, wherein the poorly soluble drug comprises one or more of glipizide, doxazosin, verapamil, prazosin, isradipine, cilostazol, nifedipine, nisoldipine, bendroflumethazide, chlorpropamide, hydrocortisone, ibuprofen, and diclofenac.

10. The monocompartment osmotic controlled drug delivery system of claim 9, wherein the poorly soluble drug comprises glipizide.

11. The monocompartment osmotic controlled drug delivery system of claim 9, wherein the poorly soluble drug comprises doxazosin.

12. The monocompartment osmotic controlled drug delivery system of claim 9, wherein the poorly soluble drug comprises cilostazol.

13. The monocompartment osmotic controlled drug delivery system of claim 1, wherein the alginic acid derivative comprises one or more of alginic acid and its pharmaceutically acceptable salts, pharmaceutically acceptable esters, or other pharmaceutically acceptable derivatives.

14. The monocompartment osmotic controlled drug delivery system of claim 13, wherein the alginic acid salt comprises one or more salts of alginic acid with sodium, potassium, magnesium, calcium or ammonia.

15. The monocompartment osmotic controlled drug delivery system of claim 14, wherein the salt of alginic acid comprises sodium alginate.

16. The monocompartment osmotic controlled drug delivery system of claim 13, wherein the alginic acid ester comprises propylene glycol alginate.

17. The monocompartment osmotic controlled drug delivery system of claim 2, wherein the pharmaceutically acceptable inert excipient comprises one or more of binders, diluents, surfactants, pH modifiers, lubricants/glidants, stabilizers, plasticizers, and coloring agents.

18. The monocompartment osmotic controlled drug delivery system of claim 2, wherein the semipermeable membrane comprises one or more of semipermeable membrane-forming polymers and one or more coating additives.

19. The monocompartment osmotic controlled drug delivery system of claim 18, wherein the semipermeable membrane-forming polymer comprises one or more of cellulose derivatives, cellulose acetate, cellulose triacetate, agar acetate, amylose acetate, cellulose acetate ethyl carbamate, cellulose acetate phthalate, cellulose acetate methyl carbamate, cellulose acetate succinate, cellulose acetate dimethylaminoacetate, cellulose acetate ethyl carbonate, cellulose acetate chloroacetate, cellulose acetate ethyl oxalate, cellulose acetate methyl sulphonate, cellulose acetate butyl sulphonate, cellulose acetate propionate, cellulose acetate diethylamino-acetate, cellulose acetate octate, cellulose acetate laurate, cellulose acetate p-toluenesulphonate, cellulose acetate butyrate, polymeric epoxides, copolymers of alkylene oxides and alkyl glycidyl ethers, polyglycols, polylactic acid derivatives, and copolymers of acrylic acid ethyl ester and methacrylic acid methyl ester.

20. The monocompartment osmotic controlled drug delivery system of claim 19, wherein the cellulose derivative comprises cellulose acetate.

21. The monocompartment osmotic controlled drug delivery system of claim 20, wherein the semipermeable membrane-forming polymer comprises a combination of cellulose acetates having different degrees of acetylation.

22. The monocompartment osmotic controlled drug delivery system of claim 18, wherein the coating additives comprises one or more of flux enhancers and pharmaceutically acceptable inert excipients.

23. The monocompartment osmotic controlled drug delivery system of claim 22, wherein the flux enhancer comprises one or more of hydroxymethyl cellulose, hydroxypropyl methylcellulose, polyethylene glycol, hydroxypropylcellulose, propylene glycol, and polyvinylpyrrolidone.

24. The monocompartment osmotic controlled drug delivery system of claim 23, wherein the flux enhancer comprises hydroxypropyl methylcellulose.

25. The monocompartment osmotic controlled drug delivery system of claim 23, wherein the flux enhancer comprises polyethylene glycol.

26. The monocompartment osmotic controlled drug delivery system of claim 3, wherein the osmotic agent comprises one or more of water soluble salts of inorganic acids, water soluble salts of organic acids, non ionic organic compounds having high water solubility, water-soluble amino acids, urea, and urea derivatives.

27. The monocompartment osmotic controlled drug delivery system of claim 26, wherein the one or more water soluble salts of inorganic acids comprises magnesium chloride, magnesium sulfate, lithium chloride, sodium chloride, potassium chloride, lithium hydrogen phosphate, sodium hydrogen phosphate, potassium hydrogen phosphate, lithium dihydrogen phosphate, sodium dihydrogen phosphate, and potassium dihydrogen phosphate.

28. The monocompartment osmotic controlled drug delivery system of claim 26, wherein the water soluble salts of organic acids comprise one or more of sodium acetate, potassium acetate, magnesium succinate, sodium benzoate, sodium citrate, and sodium ascorbate.

29. The monocompartment osmotic controlled drug delivery system of claim 26, wherein the non ionic organic compounds having high water solubility comprise one or more carbohydrate, wherein carbohydrate comprises one or more of mannitol, sorbitol, arabinose, ribose, xylose, glucose, fructose, mannose, galactose, sucrose, maltose, lactose, and raffinose.

30. The monocompartment osmotic controlled drug delivery system of claim 26, wherein the water-soluble amino acids comprises one or more of glycine, leucine, alanine, and methionine.

31. The monocompartment osmotic controlled drug delivery system of claim 26, wherein the osmotic agent comprises sorbitol.

32. The monocompartment osmotic controlled drug delivery system of claim 26, wherein the osmotic agent comprises lactose.

33. The monocompartment osmotic controlled drug delivery system of claim 10, wherein the poorly soluble drug comprises glipizide present at approximately 2.5 mg.

34. The monocompartment osmotic controlled drug delivery system of claim 10, wherein the poorly soluble drug comprises glipizide present at approximately 5 mg.

35. The monocompartment osmotic controlled drug delivery system of claim 10, wherein the poorly soluble comprise glipizide present at approximately 10 mg.

36. A process for the preparation of a monocompartment osmotic controlled drug delivery device, comprising the steps of:
blending a poorly soluble drug, at least one alginic acid derivative, and at least one pharmaceutically acceptable inert excipient; and
compressing the blend into a compact core;
enclosing the core with a solution/dispersion of an enclosing composition comprising one or more semipermeable membrane-forming polymers and other coating additives; and
forming at least one passageway in the semipermeable membrane.

37. The process of claim 36, further comprising granulating the blend with a binder before compressing the blend into a compact core.

38. The process of claim 36, further comprising blending at least one alginic acid derivative with the blend.

39. The process of claim 36, wherein the solution/dispersion of the enclosing composition is made in a solvent comprising one or more of dichloromethane, isopropyl alcohol, acetone, methanol, ethanol, and water.

40. The process of claim 37, wherein the granulation is made in a solvent comprising one or more of dichloromethane, isopropyl alcohol, acetone, methanol, ethanol, and water.

41. A method of achieving controlled delivery of a poorly soluble drug over a period of at least 4 hours, the method comprising providing a monocompartment osmotic controlled drug delivery system comprising a poorly soluble drug and at least one alginic acid derivative.
